(19) United States
(12) Patent Application Publication
(10) Pub. No.: US 2012/0004567 A1
(43) Pub. Date: Jan. 5, 2012

(54) RHYTHM CORRELATION DIAGNOSTIC MEASUREMENT

(76) Inventors: LeAnne M. Eberle, Mahtomedi, MN (US); David L. Perschbacher, Coon Rapids, MN (US)

(21) Appl. No.: 13/166,124

(22) Filed: Jun. 22, 2011

Related U.S. Application Data
(60) Provisional application No. 61/360,740, filed on Jul. 1, 2010.

(51) Int. Cl. A61B 5/0402

(52) U.S. Cl. 600/515

(57) ABSTRACT

An ambulatory medical device includes a cardiac activity sensing circuit and a processing circuit. The processing circuit includes a correlation circuit and a rhythm discrimination circuit. The correlation circuit generates an indication of correlation between each of at least a portion of the cardiac depolarizations and a stored template representative of a normal sinus rhythm. The rhythm discrimination circuit is configured to compare the indications of correlation to a specified correlation threshold value, classify the information representative of cardiac activity as a specific cardiac rhythm using the comparison, and identify at least one indication of correlation that determines the classification. The processing circuit provides the identified indication of correlation to a user or process.

### CROSS-REFERENCE TO RELATED APPLICATIONS

This application claims the benefit of U.S. Provisional Application No. 61/360,740, filed on Jul. 1, 2010, under 35 U.S.C. §119(e), which is incorporated herein by reference in its entirety.

### BACKGROUND

Cardioverter defibrillators are medical devices that deliver an electrical shock to the heart via electrodes to terminate arrhythmias. The devices may use the same or a different set of electrodes to monitor electrical heart activity within a patient.

Automated external defibrillators (AEDs) include surface electrodes that are applied to a patient by a paramedic or other trained personnel. Wearable cardioverter defibrillators (WCDs) are personal external monitors that are worn by the patient and contain surface electrodes. The surface electrodes are arranged to provide one or both of monitoring surface electrocardiograms (ECGs) and delivering cardioverter and defibrillator shock therapy.

Implantable cardioverter defibrillators (ICDs) include implantable electrodes. The electrodes are connected to sense amplifiers to provide internal monitoring of a patient's condition. ICDs may include one or more sensors to monitor one or more other internal patient parameters. In other examples, the ICDs are included in a cardiac function management device (CFM) that provides a combination of device capabilities such as pacemaker therapy and cardiac resynchronization therapy (CRT).

Additionally, some medical devices detect events by monitoring electrical heart activity signals. These events can include heart chamber electrical depolarization and the subsequent expansions and contractions. By monitoring cardiac signals indicative of expansions or contractions, medical devices can detect abnormally rapid heart rate, such as tachyarrhythmia. Tachyarrhythmia includes ventricular tachycardia (VT) which originates from the ventricles. Tachyarrhythmia also includes rapid and irregular heart rate, or fibrillation, including ventricular fibrillation (VF). Abnormally rapid heart rate can also be due to supraventricular tachycardia (SVT). SVT is less dangerous to the patient than VT or VF. SVT includes arrhythmias such as atrial tachycardia, atrial flutter, and atrial fibrillation. A rapid heart rate can also be due to sinus tachycardia, which is a normal response to, for example, exercise or an elevated emotional state.

Typically, cardioverter defibrillators detect tachyarrhythmia by first detecting a rapid heart rate. When detected, ventricular tachyarrhythmia can be terminated using high-energy cardioversion/defibrillation shock therapy. Other detection methods in addition to fast rate detection are used to reduce the incidence of inappropriate shocks. It is important for cardioverter defibrillators to quickly and accurately classify sensed rhythms or arrhythmias and deliver the appropriate therapy.

### Overview

This document relates generally to systems, devices, and methods for classifying cardiac rhythms. A system example includes an ambulatory medical device. The ambulatory medical device includes a cardiac activity sensing circuit and a processing circuit. The processing circuit includes a correlation circuit and a rhythm discrimination circuit. The correlation circuit generates an indication of correlation between each of at least a portion of the cardiac depolarizations and a stored template representative of a normal sinus rhythm. The rhythm discrimination circuit is configured to compare the indications of correlation to a specified correlation threshold value, classify the information representative of cardiac activity as a specific cardiac rhythm using the comparison, and identify at least one indication of correlation that determines the classification. The processing circuit provides the identified indication of correlation to a user or process.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an illustration of portions of a system that uses an implantable medical device (IMD).

FIG. 2 is a flow diagram of an example of a method of classifying a sensed cardiac rhythm.

FIG. 3 is a block diagram of portions of an example of a system to classify a sensed or detected intrinsic cardiac rhythm.

FIG. 4 shows a conceptualized cardiac signal segment and a template signal segment.

FIGS. 5A and 5B are representations of correlating a sampled cardiac signal segment with a template.

FIG. 6 shows an example of a dialog screen of a display.

FIG. 7 shows another example of a dialog screen of a display.

FIG. 8 shows an example of displaying indications of correlations with a segment of a sensed cardiac signal.

FIG. 9 shows another example of displaying indications of.

### DETAILED DESCRIPTION

A medical device may include one or more of the features, structures, methods, or combinations thereof described herein. For example, a cardiac monitor or a cardiac stimulator may be implemented to include one or more of the advantageous features or processes described below. It is intended that such a monitor, stimulator, or other implantable, partially implantable, ambulatory, or wearable device need not include all of the features described herein, but may be implemented to include selected features that provide for unique structures or functionality. Such a device may be implemented to provide a variety of therapeutic or diagnostic functions.

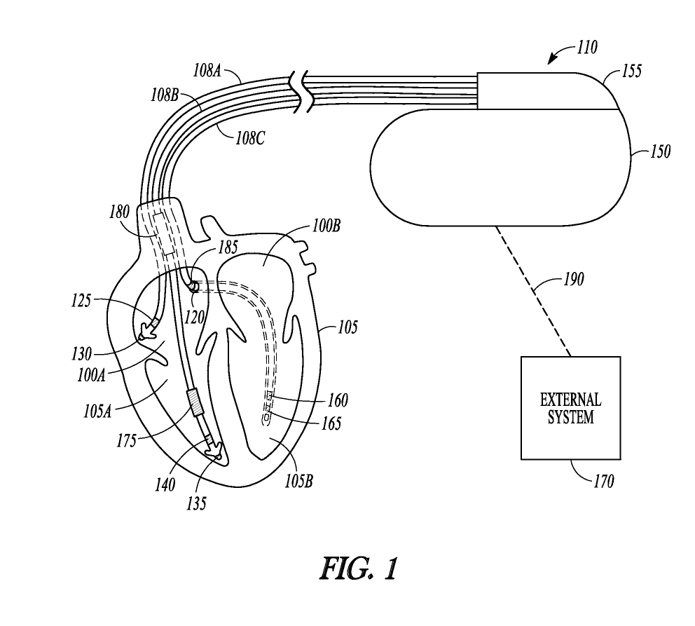

This document discusses, among other things, systems, devices, and methods for discriminating heart rhythms. An ambulatory medical device includes medical devices that can be worn, implanted, or partially implanted. FIG. 1 is an illustration of portions of a system that uses an implantable medical device (IMD) 110. Examples of IMD 110 include, without limitation, a cardioverter defibrillator, a pacer, a cardiac resynchronization therapy (CRT) device, or a combination of such devices. The system also typically includes an IMD programmer or other external device 170 that communicates wireless signals 190 with the IMD 110, such as by using radio frequency (RF) or other telemetry signals.

The IMD 110 is coupled by one or more leads 108A-C to heart 110. Cardiac leads 108A-C include a proximal end that is coupled to IMD 110 and a distal end, coupled by electrical contacts or “electrodes” to one or more portions of a heart 105. The electrodes typically deliver cardioversion, defibrillation, pacing, or resynchronization therapy, or combinations thereof to at least one chamber of the heart 105. The electrodes may be electrically coupled to sense amplifiers to sense electrical cardiac signals.

Heart 105 includes a right atrium 100A, a left atrium 100B, a right ventricle 105A, a left ventricle 105B, and a coronary sinus 120 extending from right atrium 100A. Right atrial (RA) lead 108A includes electrodes (electrical contacts, such as ring electrode 125 and tip electrode 130) disposed in an atrium 100A of heart 105 for sensing signals, or delivering pacing therapy, or both, to the atrium 100A.

Right ventricular (RV) lead 108B includes one or more electrodes, such as tip electrode 135 and ring electrode 140, for sensing signals, delivering pacing therapy, or both sensing signals and delivering pacing therapy. Lead 108B optionally also includes additional electrodes, such as for delivering atrial cardioversion, atrial defibrillation, ventricular cardioversion, ventricular defibrillation, or combinations thereof to heart 105. Such electrodes typically have larger surface areas than pacing electrodes in order to handle the larger energies, voltages, and currents involved in defibrillation. Lead 108B optionally provides resynchronization therapy to the heart 105. Resynchronization therapy is typically delivered to the ventricles in order to better synchronize the timing of depolarizations between ventricles.

The IMD 110 may include a third cardiac lead 108C attached to the IMD 110 through the header 155. The third cardiac lead 108C includes ring electrodes 160 and 165 placed in a coronary vein lying epicardially on the left ventricle (LV) 105B via the coronary vein. The third cardiac lead 108C may include a ring electrode 185 positioned near the coronary sinus (CS) 120.

Lead 108B may include a first defibrillation coil electrode 175 located proximal to tip and ring electrodes 135, 140 for placement in a right ventricle, and a second defibrillation coil electrode 180 located proximal to the first defibrillation coil 175, tip electrode 135, and ring electrode 140 for placement in the superior vena cava (SVC). In some examples, high-energy shock therapy is delivered from the first or RV coil 175 to the second or SVC coil 180. In some examples, the SVC coil 180 is electrically tied to an electrode formed on the hermetically-sealed IMD housing or can 150. This improves defibrillation by delivering current from the RV coil 175 more uniformly over the ventricular myocardium. In some examples, the therapy is delivered from the RV coil 175 only to the electrode formed on the IMD can 150.

Note that although a specific arrangement of leads and electrodes are shown the illustration, the present methods and systems will work in a variety of configurations and with a variety of electrodes. Other forms of electrodes include meshes and patches which may be applied to portions of heart 105 or which may be implanted in other areas of the body to help “steer” electrical currents produced by IMD 110. The IMDs may be configured with a variety of electrode arrangements, including transvenous, endocardial, or epicardial electrodes (e.g., intrathoracic electrodes), or subcutaneous, non-intrathoracic electrodes, such as can, header, or indifferent electrodes, or subcutaneous array or lead electrodes (e.g., non-intrathoracic electrodes). WCDs and AEDs may contain surface electrode arrangements for one or both of monitoring surface electrocardiograms (ECGs) and delivering cardioverter and defibrillator shock therapy. Implantable electrode arrangements using electrodes implanted in or near a heart chamber provide for monitoring internal electrograms (egrams).

Egrams may be sensed using electrodes to deliver electrical pacing therapy. The arrangement of such electrodes is sometimes called a rate channel (e.g., electrodes 140 and 135 in FIG. 1). Egrams may also be sensed using electrodes to deliver higher energy shock therapy such as cardioversion or defibrillation shock therapy. This arrangement of electrodes sometimes called a shock channel (e.g., electrode 180 and an electrode formed on IMD can 150). ECGs can be sensed by a wearable device using electrodes to sense cardiac activity (rate channel), or by electrodes to deliver shock therapy (shock channel). Monitoring of electrical signals related to cardiac activity may provide early, if not immediate, diagnosis of cardiac disease.

It can be useful to a caregiver to know when a sensed heart rhythm is, or is not, of a certain type. To identify a heart rhythm, a medical device may compare a sensed intrinsic cardiac signal to a template signal waveform or template waveform segment. A template can be thought of as a snapshot of a cardiac signal of the subject (e.g., a snapshot of normal sinus rhythm). A medical device may calculate the similarity of the sensed waveform to the template to identify the heart rhythm. The medical device may then base a device therapy or diagnostic decision (e.g., begin storing egrams) on the calculation. It can be useful to the caregiver to know the details of the device decision-making. In this way, the caregiver can adjust the decision-making rules of the medical device to optimize operation of the medical device.

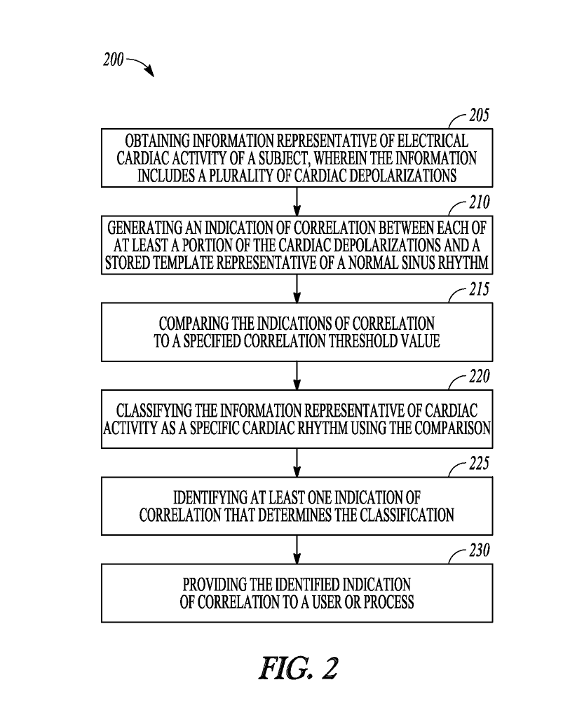

FIG. 2 is a flow diagram of an example of a method 200 of classifying a sensed cardiac rhythm. At block 205, information representative of electrical cardiac activity of a subject is obtained using an ambulatory medical device. The information includes a plurality of cardiac depolarizations. In some examples, the information includes a sensed cardiac signal, such as a cardiac signal sensed in or near a ventricle or atrium.

At block 210, an indication of correlation is generated between each of at least a portion of the cardiac depolarizations and a stored template representative of a normal sinus rhythm. In some examples, the indication of correlation is a calculation of a measure of similarity between the cardiac information and a stored template. The template can be a representation of a rhythm of interest (e.g., a normal sinus rhythm of the subject).

At block 215, the generated indications of correlation are compared to a specified correlation threshold value. The correlation threshold value can be programmed into the ambulatory medical device. At block 220, the information representative of cardiac activity is classified as a specific cardiac rhythm using the comparison of the indications of correlation to the specified correlation threshold value.

At block 225, at least one indication of correlation is identified that determines the classification. This identified indication of correlation may be the indication of correlation that leads the device to make a decision about the sensed rhythm.

In some examples, indications of correlation are generated over multiple segments of a sensed cardiac signal, and are compared to the threshold correlation value. The segments may or may not overlap. The device may make a decision over each segment. In this case, an indication of correlation may be identified for each segment. In some examples, a central tendency (e.g., a median or an average) of the indications is calculated.

At block 230, the identified indication of correlation is provided to a user or process. The identified indication of correlation can be useful to a caregiver to understand why the comparison to the template caused the medical device to operate in a certain way.

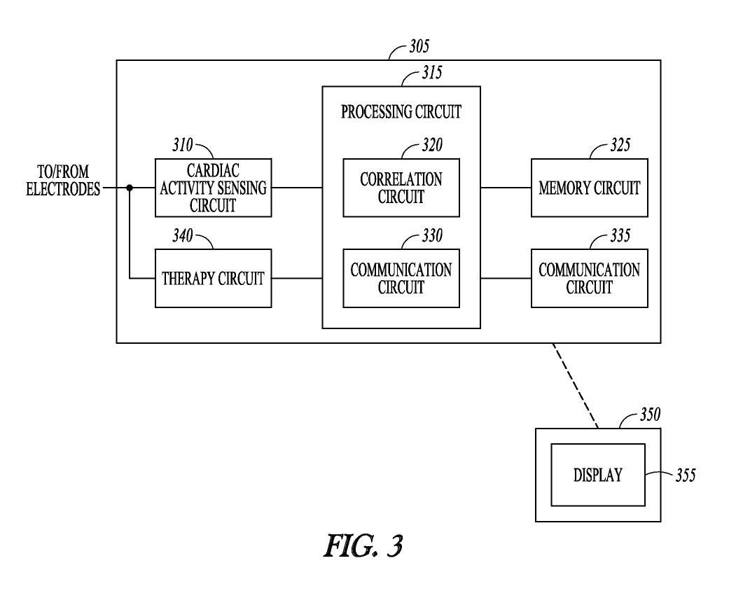

FIG. 3 is a block diagram of portions of an example of a system 300 to classify a sensed or detected intrinsic cardiac rhythm. The system 300 includes an ambulatory medical device 305. In some examples, the ambulatory medical device 305 is an IMD. In some examples, the ambulatory medical device 305 is a partially implantable device. In some examples, the ambulatory medical device 305 is a wearable device.

The ambulatory medical device 305 includes a cardiac activity sensing circuit 310. The cardiac activity sensing circuit 310 obtains information representative of electrical cardiac activity of a subject. In some examples, the cardiac activity sensing circuit 310 senses intrinsic cardiac signals when it is communicatively coupled to electrodes. For instance, the cardiac activity sensing circuit 310 can sense an intrinsic ventricular cardiac signal when communicatively coupled to electrodes for placement in or near ventricle. In some examples, the cardiac activity sensing circuit 310 includes a sampling circuit to sample a cardiac signal. The sampling circuit may include an analog to digital converter (ADC) to convert a sensed cardiac signal to discrete digital values. The cardiac activity information may include a sampled intrinsic cardiac activity signal. In some examples, the information includes a plurality of cardiac depolarizations. At least a portion of the depolarizations may be compared to a template to determine what rhythm the cardiac depolarizations represent.

The ambulatory medical device 305 also includes a processing circuit 315 communicatively coupled to the cardiac activity sensing circuit 310. The communicative coupling allows electrical signals to be communicated between the cardiac activity sensing circuit 310 and the processing circuit 315 even though there may be intervening circuitry. The processing circuit 315 may include a processor such as a microprocessor, a digital signal processor, application specific integrated circuit (ASIC), microprocessor, or other type of processor, interpreting or executing instructions in software or firmware. The processing circuit 315 may include other circuits or sub-circuits to perform the functions described. These circuits may include software, hardware, firmware or any combination thereof. Multiple functions can be performed in one or more of the circuits as desired.

The processing circuit 315 includes a correlation circuit 320 that generates an indication of correlation between each of at least a portion of the cardiac depolarizations and a stored template representative of a specified cardiac rhythm. The template may be stored in a memory circuit 325 integral to or communicatively coupled to the processing circuit 315. In some examples, the template is representative of normal sinus rhythm (NSR). In some examples, multiple templates are stored in the memory for multiple types of cardiac rhythms.

In some examples, the processing circuit 315 includes a template circuit configured to generate one or more templates of cardiac signals sensed from the subject. An approach for generating electrical cardiac signal templates using a snapshot of the subject's conducted heart beats is described in Kim et al., U.S. Pat. No. 6,708,058, entitled “Normal Cardiac Rhythm Template Generation System and Method,” filed Apr. 30, 2001, which is incorporated herein by reference in its entirety.

In some examples, the indication of correlation is calculated for each depolarization or a portion of the depolarizations in the cardiac activity information. In some examples, the indication of correlation is calculated on a sampled cardiac signal that is averaged over a time window having a specified number of cardiac depolarizations or a specified duration of time.

The arrhythmia of the subject may include skipped heart beats. In this case, there may be an extended interval of time in the cardiac activity information without a depolarization. This may correspond to a situation where an escape interval timer circuit of the ambulatory medical device 305 times out without a sensed intrinsic depolarization. In certain examples, such an interval or timeout is included in the correlation calculation as a “no-sense event.” In certain examples, a no-sense event is excluded from the correlation calculation.

In certain examples, the indication of correlation includes a calculated correlation coefficient (CC), or a feature correlation coefficient (FCC). The CC calculated by the correlation circuit 320 indicates the degree of similarity between a shape of the segment of the cardiac signal that includes the located cardiac features and a shape of the template segment. Examples of calculating correlation coefficients are discussed in the previously mentioned U.S. Pat. No. 6,708,058.

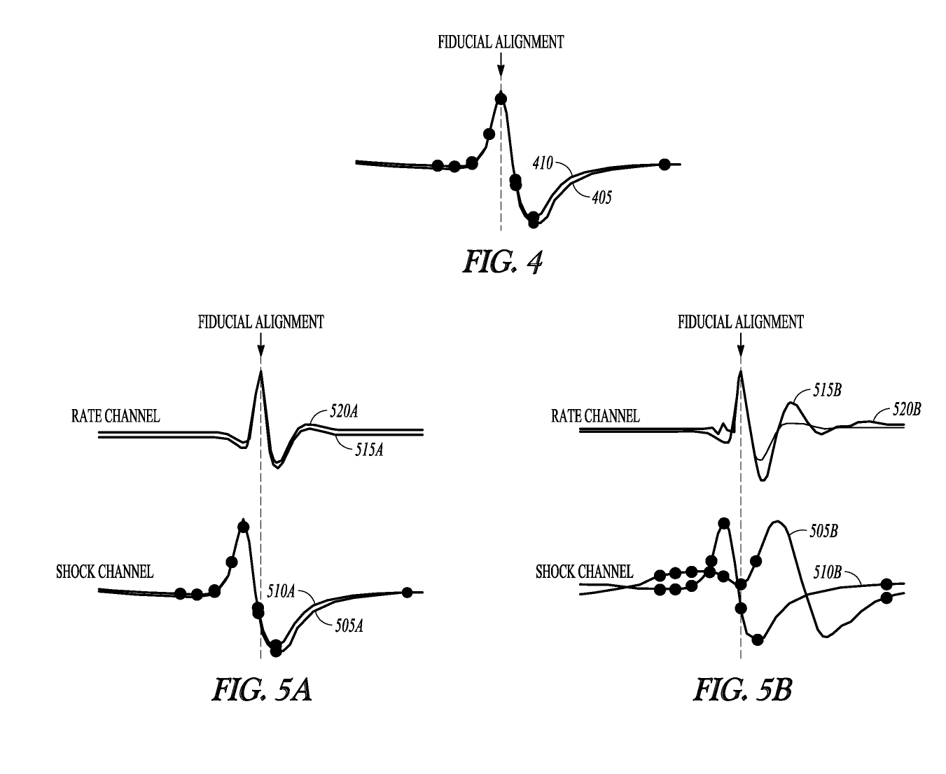

FIG. 4 shows a conceptualized cardiac signal segment 405 (i.e., not real data) and a template signal segment 410. In some examples, the correlation circuit identifies a fiducial feature in the cardiac signal. In some examples, the fiducial feature includes an R-wave peak in cardiac activity information. An R-wave refers to the first typically positive deflection in the QRS complex of an ECG or egram.

The correlation circuit 320 aligns the fiducial feature of the cardiac signal segment 405 with the corresponding feature in the template segment 410. In some examples, the correlation circuit 320 then uses N comparison points (x₁, x₂, . . . x_{N}: y₁, y₂, . . . y_{N}) to calculate the CC. In certain examples, N=8 and the CC is calculated by

$$\frac{{( 8 {\sum }_{i = 1}^{8} {x}_{i} {y}_{i} - ( {\sum }_{i = 1}^{8} {x}_{i} ) ( {\sum }_{i - 1}^{8} {y}_{i} ) )}^{2}}{( 8 {\sum }_{i = 1}^{8} {x}_{i}^{2} - {( {\sum }_{i = 1}^{8} {x}_{i} )}^{2} ( 8 {\sum }_{i = 1}^{8} {y}_{i}^{2} - {( {\sum }_{i = 1}^{8} {y}_{i} )}^{2} )} .$$

The correlation circuit 320 may use one signal segment to identify the fiducial feature and a different signal segment to calculate the CC. In some examples, the cardiac activity sensing circuit 310 includes multiple sensing channels, such as a first sensing channel configured to provide a first cardiac signal, and a second sensing channel configured to provide a second sensing channel. For instance, a cardiac signal sensed with a shock channel may provide more morphology information than a cardiac signal sensed using a rate channel. The correlation circuit 320 may identify the fiducial feature using the first cardiac signal (e.g., from a rate channel) and calculate the CC using the second cardiac signal (e.g., from a shock channel).

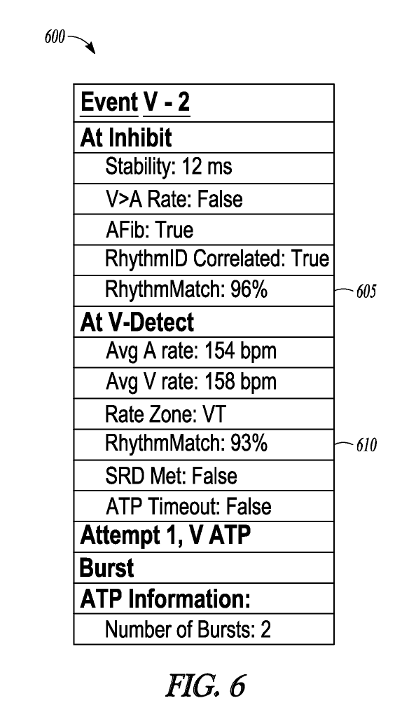

This is shown in FIGS. 5A and 5B. The signals 505A, 505B represents a signal sensed using a shock channel and the template 510A, 510B is for a shock channel comparison. The signal 515A, 515B represents a cardiac signal sensed using a rate channel and the template 520A, 520B is for a rate channel comparison. The rate channel signal is sensed in a known relationship to the shock channel signal (e.g., sensed at the same time). The correlation circuit 320 aligns the fiducial feature in the rate channel signal 515A, 515B with the corresponding fiducial feature in the rate channel template 520A, 520B. Because the timing relationship to the shock channel is known, the correlation circuit 320 is able to align shock channel signal 505A, 505B and shock channel template 510A, 510B with the fiducial feature. The correlation circuit 320 then calculates an indication of correlation (e.g., a CC) for the shock channel.

Returning to FIG. 3, the processing circuit 315 also includes a rhythm discrimination circuit 330 that compares the indications of correlation to a specified correlation threshold value. FIG. 5A is a representation of a sampled cardiac signal segment 505A correlating well with the template. FIG. 5B is a representation where the correlation is not as good. In some examples, correlation threshold value of 90% is specified in the ambulatory medical device 305. The correlation threshold value may be a programmable parameter. If the calculated correlation is >90%, the sensed signal is deemed to correlate with the template. If the correlation ≦90%, the sensed signal is deemed to be uncorrelated.

The rhythm discrimination circuit 330 classifies the information representative of cardiac activity as a specific cardiac rhythm using the comparison of the indications of correlation. The correlation circuit 320 may calculate indications of correlation for multiple cardiac depolarizations. These cardiac depolarizations may be a succession of depolarizations in a sensed cardiac signal segment. The rhythm discrimination circuit 330 uses the multiple indications of correlation to identify the rhythm. For example, the rhythm discrimination circuit 330 may classify the cardiac activity information as NSR when X of Y cardiac depolarizations satisfy the specified correlation threshold value, where X and Y are integers and X is less than Y (e.g., X can be 3 and Y can be 10). In some examples, the numbers X and Y are programmable. In some examples, the correlation circuit 320 calculates indications of correlation for multiple cardiac depolarizations of multiple cardiac signal segments. For instance, the correlation circuit 320 may calculate indications of correlation for multiple signal segments of Y cardiac depolarizations.

The rhythm discrimination circuit 330 labels, flags, or otherwise identifies the indication of correlation that determines the classification. In the previous example, the rhythm discrimination circuit 330 identifies the Xth highest indication of correlation. This is the indication that caused the rhythm discrimination circuit 330 to make the decision about the sensed rhythm because this indication of correlation satisfied the classification criteria. In some examples, if the correlation circuit 320 calculates indications of correlation for multiple segments of Y cardiac depolarizations, the rhythm discrimination circuit 330 may use a central tendency (e.g., an average or median) of the identified indications of correlation for the segments.

The processing circuit 315 provides the identified indication of correlation to a user or process. In some examples, the process may be executing on the ambulatory medical device 305 or a second separate device. In some examples, the ambulatory medical device 305 includes a communication circuit 335 to communicate information wirelessly with a second device. An approach to communications using medical devices can be found in U.S. Pat. No. 7,664,553, “Systems and Method for Enabling Communications with Implantable Medical Devices,” filed Apr. 27, 2005, which is incorporated herein by reference in its entirety. In some examples, the second device includes a display and the identified indication of correlation can be communicated to the second device for display to a user.

The second device can be a remote device that includes an IMD programmer. In some examples, the ambulatory medical device 305 communicates with the remote device via a third device (e.g., a repeater). In some examples, the remote device is part of an advanced patient management (APM) system, and includes a server connected to a computer network such as the internet.

The described methods and devices are particularly useful in classifying tachyarrhythmia. When VT is detected, medical devices are designed to provide therapy to the patient. Cardioverter defibrillators (e.g., wearable or implantable) treat VT by delivering high energy shock therapy to the heart. Another therapy for tachyarrhythmia is anti-tachycardia pacing (ATP). ATP may be delivered by an implantable or partially implantable pacemaker or by an ICD. ATP uses lower energy pacing therapy to establish a regular rhythm in a heart. This allows the tachycardia to be converted to a normal heart rhythm without exposing the patient to high energy cardioversion/defibrillation therapy that can be painful to the patient. Providing painless therapy, such as ATP therapy, improves the patient's experience with an IMD as well as increasing the battery longevity of the devices.

Some types of tachyarrhythmia are considered to be serious enough to warrant delivering shock therapy immediately (e.g., VF or fast VT). Other types of tachyarrhythmia may be considered less urgent (e.g., slow VT, or SVT) and a medical device may be configured to first try to convert the tachyarrhythmia using a less aggressive therapy such as ATP. For some arrhythmias (e.g., SVT) a medical device may be programmed to not provide any therapy.

If a medical device incorrectly interprets a detected arrhythmia, the device may inappropriately deliver shock therapy or other therapy. Typically, the majority of inappropriate shocks are delivered when a device fails to correctly distinguish a heart arrhythmia as being SVT. Thus, it is desirable for a medical device to correctly recognize and prevent inappropriate delivery of shock therapy.

According to some examples, the rhythm discrimination circuit 330 can detect a tachyarrhythmia using the information representative of cardiac activity. For example, the rhythm discrimination circuit 330 may detect a tachyarrhythmia when intervals between depolarizations in the cardiac activity information are less than a lowest tachyarrhythmia detection threshold. When the rhythm discrimination circuit 330 detects an abnormally rapid heart rate that may indicate tachyarrhythmia, the device may use detection enhancements to further classify the arrhythmia.

In some examples, the rhythm discrimination circuit 330 performs a rhythm discrimination process that includes recurrently updating an average ventricular contraction interval (V-V interval) and determining that an average ventricular contraction rate exceeds an average atrial contraction rate (V>A) by more than a specified rate threshold value (e.g., ten beats per minute). Descriptions of systems and methods for classifying detected tachycardia based on average atrial and ventricular rates calculated from selected atrial and ventricular intervals is found in co-pending U.S. patent application Ser. No. 11/054,726, Elahi et al., entitled, “Method and Apparatus for Rate Accuracy Enhancement in Ventricular Tachycardia Detection,” filed Feb. 10, 2005, which is incorporated herein by reference in its entirety.

In some examples, the rhythm discrimination circuit 330 performs a rhythm discrimination method that includes assessing stability of the ventricular rhythm. In an example, the stability is assessed by measuring the degree of variability of R-R intervals during the tachycardia episode. The current average difference between R-R intervals is compared to a programmed stability threshold and a “shock if unstable” threshold. If the average difference is greater than the programmed thresholds, the rhythm is declared unstable. Descriptions of methods and systems to detect abnormal heart rhythms and assess the stability of a ventricular rhythm are found in Gilkerson et al., U.S. Pat. No. 6,493,579, entitled “System and Method for Detection Enhancement Programming,” filed Aug. 20, 1999, which is incorporated herein by reference in its entirety.

In some examples, the rhythm discrimination circuit 330 uses the morphology of the cardiac activity information to classify the arrhythmia. In some examples, the rhythm discrimination circuit 330 uses any combination of V>A, rate stability, and morphology analysis to classify a detected arrhythmia. To classify the morphology of the detected arrhythmia, indications of correlation are generated and the rhythm discrimination circuit 330 classifies the arrhythmia using the indications.

In some examples, the correlation circuit 320 generates an indication of correlation the cardiac depolarizations in the cardiac activity information and a template representative of NSR. The rhythm discrimination circuit 330 classifies the arrhythmia as supraventricular tachycardia (SVT) when a minimum of X of Y cardiac depolarizations satisfy the specified correlation threshold value, wherein X and Y are integers and X is less than Y (e.g., X can be 3 and Y can be 10). The rhythm discrimination circuit 330 identifies the Xth highest indication of correlation to the user or process as the indication that determines the classification. The Xth highest indication of correlation is the indication that caused the device to identify the arrhythmia as SVT. If the more than a specified number of the Y beats were paced events or no-sense events (e.g., 2 out of 10 beats), the rhythm discrimination circuit 330 may not be able to identify an indication of correlation that determines a classification.

In some examples, the rhythm discrimination circuit 330 classifies the arrhythmia as VT when a minimum of Z of Y cardiac depolarizations fail to satisfy the specified correlation threshold value, wherein Z and Y are integers and Z is less than Y (e.g., Z can be 8 and Y can be 10). The rhythm discrimination circuit 330 identifies the Zth lowest indication of correlation to the user or process as the indication that determines the classification. The Zth lowest indication of correlation is the indication that caused the device to identify the arrhythmia as VT.

In some examples, the arrhythmia must also satisfy a specified time duration threshold before the rhythm discrimination circuit classifies the arrhythmia as VT. If more than Z of the Y heart beats are too fast (e.g., the beats satisfy a specified VF detection interval threshold, or the interval of the fast beats is less than or equal to a specified interval threshold, such as 260 ms), the rhythm discrimination circuit 330 may not be able to identify an indication of correlation that determines a classification.

In some examples, the ambulatory medical device 305 may include a therapy circuit 340 communicatively coupled to the processing circuit 315. The therapy circuit 340 can provide one or more of anti-tachyarrhythmia pacing (ATP) therapy or anti-tachyarrhythmia high-energy defibrillation/cardioversion shock therapy to the subject. Anti-tachyarrhythmia therapy can result in perceived discomfort or acceleration of the tachyarrhythmia to higher rates that are poorly tolerated by the patient. For this reason, the processing circuit 315 may inhibit delivery of therapy if the classification is SVT. If the classification is VT, the processing circuit 315 may initiate delivery of ATP therapy if the VT includes a relatively slow tachyarrhythmia rate before resorting to shock therapy. If the classification is VT, the processing circuit 315 may directly initiate delivery of shock therapy.

The indication of correlation that resulted in the classification of the arrhythmia can be important for a caregiver to know. The classification can cause the ambulatory medical device 305 to alter its operation, such as to determine which therapy to deliver or whether to inhibit therapy. Using the indication of correlation, the caregiver can determine the decision making criteria of the device and choose to change the operation of the device by changing the specified correlation threshold value.

FIG. 6 shows an example of a dialog screen 600 of a display for presenting the identified indication of correlation to a user. In the display, the term “RhythmMatch” shows the identified indication of correlation that caused the rhythm discrimination circuit 330 to decide on the classification of the rhythm. As shown in the example, the identified indication of correlation 605 can be a CC. For the arrhythmia episode, the dialog screen 600 shows that the detected cardiac activity correlated (“RhythmID Correlated: True”) to a template of NSR and that the processing circuit 315 inhibited delivery of therapy. The dialog screen 600 may also show the result of rhythm detection enhancements such as Rhythm Stability and V>A. In the example, the specified correlation threshold value was 95%. The rhythm discrimination circuit 330 was programmed to classify the arrhythmia as SVT when at least 3 out of 10 cardiac depolarizations of cardiac activity correlated to the template of NSR. Therefore, the identified indication of correlation (of 96%) was the third highest indication of correlation generated. The example shows that the identified indication of correlation can be recorded when therapy is inhibited in an arrhythmia episode, even if the detection criteria are never met.

The dialog screen 600 also shows that later in the episode, the arrhythmia was classified as VT. The rhythm discrimination circuit 330 was programmed to classify the arrhythmia as VT when at least 8 out of 10 cardiac depolarizations of cardiac activity did not correlate to the template of NSR. Therefore, the identified indication of correlation (of 93%) was the eighth lowest indication of correlation generated. The dialog screen also shows that the processing circuit 315 attempted to convert the VT episode with ATP.

By reviewing the identified indication of correlation, a caregiver may determine a correlation threshold value that is sufficiently above the subject's calculated correlation values for appropriate VT classification, but is still below the calculated correlation value for SVT classification. Reprogramming the correlation threshold allows the caregiver to tailor the morphology detection criterion and allow the rhythm discrimination circuit 330 of the ambulatory medical device 305 to more accurately distinguish between VT and SVT, and thereby potentially reduce delivery of inappropriate therapy. If an inappropriate therapy is delivered, recording the identified indication of correlation provides additional information to aid in determining appropriate therapy parameters.

In some examples, the ambulatory medical device 305 includes the communication circuit 335 and the ambulatory medical device 305 receives an updated correlation threshold from the second device 350. The correlation circuit 320 is configured to use the updated correlation threshold as the specified correlation threshold for subsequent correlation determination. If the template is a representation of NSR, increasing the correlation threshold makes the rhythm discrimination circuit 330 more sensitive to detection of VT and less specific for detection of SVT, and decreasing the correlation threshold makes the rhythm discrimination circuit 330 less sensitive to detection of VT and more specific for detection of SVT.

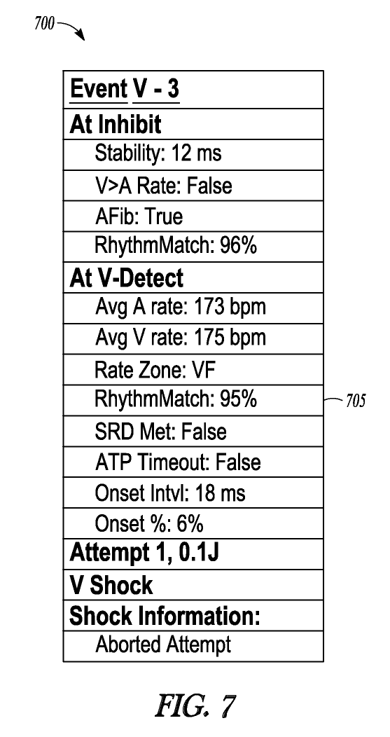

FIG. 7 shows another example of a dialog screen 700 of a display for presenting the identified indication of correlation to a user. For the episode shown, the rhythm discrimination circuit 330 classified the arrhythmia as VF based on the detected heart rate. For VF, the processing circuit 315 initiates cardioversion defibrillation shock therapy, and typically does not take into account a morphology analysis by the rhythm discrimination circuit 330. The dialog screen 700 shows that the rhythm discrimination circuit 330 may still perform the analysis, and that the identified indication of correlation 705 can still be recorded where the detection is by rate only or by rate with the rate stability detection enhancement.

By presenting the identified indication of correlation 705 to the caregiver, the caregiver may choose to reprogram the detection parameters of the rhythm discrimination circuit 330. For instance, the caregiver may raise the VF detection rate threshold. This can allow the ambulatory medical device 305 to use the morphology analysis to inhibit therapy for SVT at higher rates. In another example, the caregiver may decide to switch the detection enhancement used to prevent inappropriate therapy for sinus tachycardia (ST).

The identified indication of correlation can be calculated and recorded when arrhythmia detection is not enabled in the device. As long as a template is stored, the device can determine a correlation of a sensed rhythm.

As explained earlier, the correlation circuit 320 may calculate indications of correlation for multiple cardiac depolarizations. A user may desire to see the succession of calculated indications of correlation for the depolarizations rather than only seeing the indication that decided the classification.

According to some examples, the correlation circuit 320 generates a first indication of correlation between a first cardiac depolarization and the stored template and generates a second indication of correlation between a second cardiac depolarization and the stored template.

The processing circuit 315 provides, to a user or process: the first and second indications of correlation and the information representative of the first and second cardiac depolarizations. The processing circuit 315 also provides information relating the first indication and the second indication to the first cardiac depolarization and the second cardiac depolarization respectively. This way the indications can be related to cardiac events in the display. In some examples, the information relating the first and second indications and the first and second cardiac depolarizations includes a timestamp stored for one or both of an indication of correlation and its corresponding depolarization.

In some examples, the ambulatory medical device 305 (e.g., if the device is wearable) includes a display for displaying the first and second indications and the first and second depolarizations. In some examples, system 300 includes a second separate device that communicates with the ambulatory medical device 305 and the second device 350 includes a display 355. In certain examples, the devices include wireless interfaces and the communication is wireless such as by near field inductive telemetry, or by far-field radio frequency (RF) communication. In certain examples, the devices include wired interfaces (e.g., a wearable ambulatory medical device with a serial (USB) port).

The first and second indications can be displayed with the depolarizations, including substantially aligning the first indication with the representation of the first cardiac depolarization on the display and substantially aligning the second indication with the representation of the second cardiac depolarization on the display.

In some examples, the indications of correlation are calculated and displayed in real time. If the display is on the second device 350, the processing circuit 315 communicates the first and second indications of correlation and the information representative of the first and second cardiac depolarizations to the second device for display. Because there may be a significant lag in the calculation of the indications of correlation, the calculated indications may be stored in a buffer together with information (e.g., a timestamp) relating the first indication and the second indication to the first cardiac depolarization and the second cardiac depolarization. This information allows the display to be correctly constructed with the indications of correlation correctly aligned with cardiac events.

In some examples, the indications of correlation are calculated and stored for later display. The memory circuit 325 stores, for subsequent display, the first and second indications of correlation, the information representative of the first and second cardiac depolarizations, and the information relating the first indication and the second indication to the first cardiac depolarization and the second cardiac depolarization.

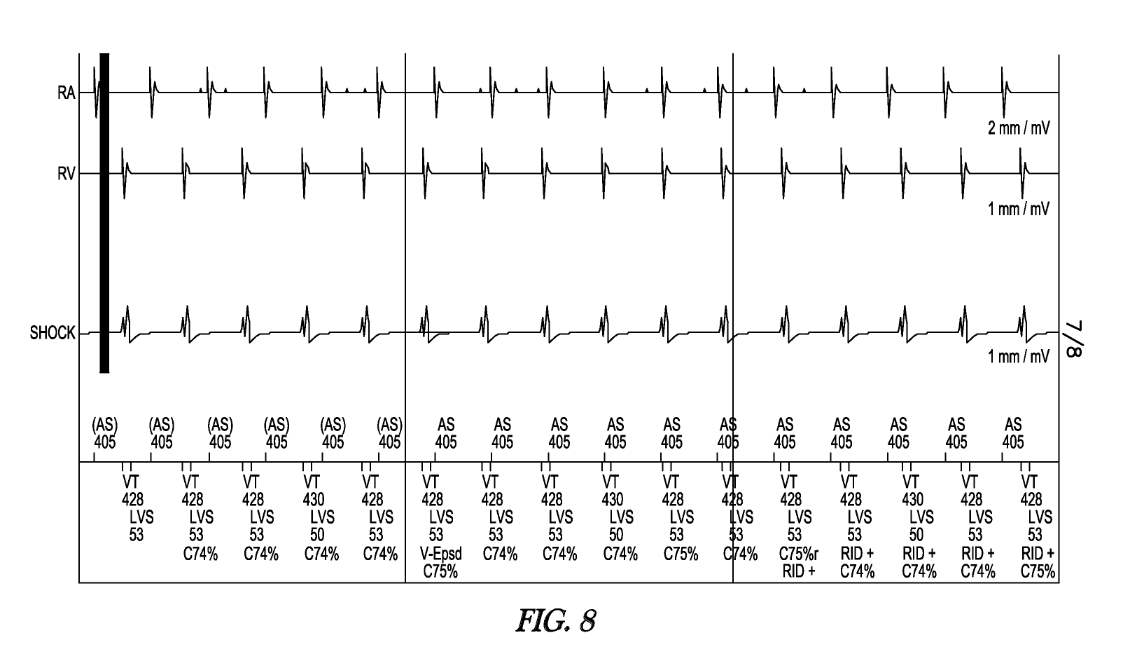

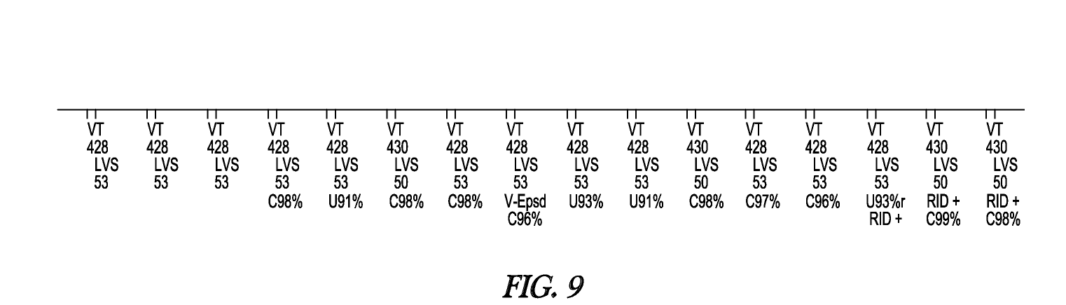

FIG. 8 shows an example of displaying indications of correlations with a segment of a sensed cardiac signal. Although the approach has been discussed for only first and second depolarizations, the Figure shows that the indications can be displayed for a succession of sensed cardiac events in association with those events. In the example shown, the specified correlation threshold value is set to 74%. The indications can be referred to as “markers” and can be displayed together with egrams from one or more of an atrial sense channel, a ventricular sense channel, and a shock channel. FIG. 9 shows an example where the indications of correlation are displayed as markers only, without any associated egrams.

In some examples, the indications of correlation are available for substantially all sensed beats. By displaying the indications of correlation, a caregiver may become more familiar with the decision making process that rhythm discrimination circuit 330 uses to classify a sensed rhythm. Reviewing one or both of the markers and egram waveforms may help a caregiver program or reprogram arrhythmia detection enhancements to reduce delivery of inappropriate therapy. The beat-to-beat indications of correlation may help the caregiver properly set the threshold correlation value for the subject for proper classification of VT and SVT.

Additionally, displaying the beat-to-beat indications of correlation may help identify SVT beats with a high correlation value that are in the VF detection zone and may help the caregiver determine when the VF rate or interval detection threshold is set too low. However, in some examples, the morphology analysis by the rhythm discrimination circuit 330 and the calculation of indications of correlation can be enabled without enabling arrhythmia detection enhancements.

In some examples, a command is entered into the second device 350 via a user interface to set the mode of displaying the indications of correlation as either multiple indications or only the deciding indication. If at least a portion of the ambulatory medical device 305 is external (e.g., wearable or only partially implantable), the command may be entered into a user interface of the ambulatory medical device 305 for display on that device.

In some examples, indications of correlation include calculated measures of correlation (e.g., CCs). The correlation circuit 320 is configured to calculate a first measure of correlation of the first cardiac depolarization to the stored template and calculate a second measure of correlation of the second depolarization to the stored template. The first and second indications of correlation include the first measured correlation and the second measured correlation respectively. An example is shown in FIGS. 8 and 9 where the indications of correlation include a CC which is a percentage of correlation to the template.

In some examples, indications of correlation include a result of the comparison. The correlation circuit 320 compares the first and second measures of correlation to a specified correlation threshold value. The first and second indications include a result of the comparison of the first measured correlation and a result of the comparison of the second measured correlation respectively. An example is shown in FIGS. 8 and 9 as an indication of correlated (e.g., “C”) or an indication of uncorrelated (e.g., “U”). The indications of correlation may include one or both of a measure of correlation and a result of a correlation comparison.

### Conclusion

Displaying the waveform correlation markers with a tachyarrhythmia episode data and/or the identified indication of correlation may reinforce the safety and effectiveness of tachyarrhythmia detection enhancements. The waveform correlation markers and identified correlation may also provide caregivers the information they need to properly program an ambulatory medical device to discriminate between VT and SVT for an individual patient. This may potentially reduce deliveries of inappropriate therapy.

In example 1, a system includes an ambulatory medical device. The ambulatory medical device optionally includes a cardiac activity sensing circuit configured to obtain information representative of electrical cardiac activity of a subject. The information optionally includes a plurality of cardiac depolarizations. The ambulatory medical device includes a processing circuit communicatively coupled to the cardiac activity sensing circuit. The processing circuit includes a correlation circuit configured to generate an indication of correlation between each of at least a portion of the cardiac depolarizations and a stored template representative of a normal sinus rhythm. The processing circuit also includes a rhythm discrimination circuit configured to compare the indications of correlation to a specified correlation threshold value, classify the information representative of cardiac activity as a specific cardiac rhythm using the comparison, and identify at least one indication of correlation that determines the classification. The processing circuit is configured to provide the identified indication of correlation to a user or process.

In example 2, the rhythm discrimination circuit of example 1 can optionally be configured to detect an arrhythmia using the information representative of cardiac activity, classify the arrhythmia as supraventricular tachycardia (SVT) when a minimum of X of Y cardiac depolarizations satisfy the specified correlation threshold value, wherein X and Y are integers and X is less than Y, and identify the Xth highest indication of correlation as the indication that determines the classification.

In example 3, the rhythm discrimination circuit of one or any combination of examples 1 and 2 can optionally be configured to detect an arrhythmia using the information representative of cardiac activity, classify the arrhythmia as ventricular tachycardia when a minimum of Z of Y cardiac depolarizations fail to satisfy the specified correlation threshold value, wherein Z and Y are integers and Z is less than Y, and identify the Zth lowest indication of correlation as the indication that determines the classification.

In example 4, the correlation circuit of one or any combination of examples 1-3 can optionally be configured to generate a first indication of correlation between a first cardiac depolarization and the stored template and generate a second indication of correlation between a second cardiac depolarization and the stored template. The processing circuit can optionally be configured to provide, to a user or process: the first and second indications of correlation, the information representative of the first and second cardiac depolarizations, and information relating the first indication and the second indication to the first cardiac depolarization and the second cardiac depolarization respectively.

In example 5, the ambulatory medical device of one or any combination of examples 1-4 can optionally include a memory circuit integral to, or communicatively coupled to, the processing circuit. The memory circuit is configured to store, for subsequent display, the first and second indications of correlation, the information representative of the first and second cardiac depolarizations, and the information relating the first indication and the second indication to the first cardiac depolarization and the second cardiac depolarization.

In example 6, the ambulatory medical device of one or any combination of examples 1-5 can optionally include a communication circuit communicatively coupled to the processing circuit, wherein the communication circuit is configured to communicate information with a second device. The processing circuit can optionally be configured to communicate, to the second device for display, the first and second indications of correlation, the information representative of the first and second cardiac depolarizations, and the information relating the first indication and the second indication to the first cardiac depolarization and the second cardiac depolarization.

In example 7, the subject matter of one or any combination of examples 1-6 can optionally include a second device configured for communicating with the ambulatory medical device and including a display. The second device can optionally be configured to display the first and second indications, including substantially aligning the first indication with the representation of the first cardiac depolarization on the display and substantially aligning the second indication with the representation of the second cardiac depolarization on the display.

In example 8, the correlation circuit of one or any combination of examples 1-7 can optionally be configured to calculate a first measure of correlation of the first cardiac depolarization to the stored template and calculate a second measure of correlation of the second depolarization to the stored template, and wherein the first and second indications of correlation include the first measured correlation and the second measured correlation respectively.

In example 9, the correlation circuit of one or any combination of examples 1-8 can optionally be configured to calculate a first measure of correlation of the first cardiac depolarization to the stored template, calculate a second measure of correlation of the second cardiac depolarization to the stored template, compare the first and second measures of correlation to a specified correlation threshold value. The first and second indications include a result of the comparison of the first measured correlation and a result of the comparison of the second measured correlation, respectively.

In example 10, the subject matter of one or any combination of examples 1-9 can optionally include a second device configured for communicating with the ambulatory medical device and including a display. The rhythm discrimination circuit can optionally be configured to apply at least one rhythm detection enhancement method to classify the rhythm. The second device can optionally be configured to display the identified indication of correlation with one or more of the rhythm classification and a result of the rhythm detection enhancement method.

In example 11, the ambulatory medical device of one or any combination of example 1-10 optionally includes a communication circuit communicatively coupled to the processing circuit. The communication circuit is optionally configured to receive an updated correlation threshold from a second device, and the correlation circuit is optionally configured to use the updated correlation threshold as the specified correlation threshold for subsequent correlation determination.

Example 12 can include, or can optionally be combined with the subject matter of any one or any combination of examples 1-11 to include subject matter (such as a method, a means for performing acts, or a machine readable medium including instructions that, when performed by the machine, cause the machine to perform acts) comprising obtaining information representative of electrical cardiac activity of a subject using an ambulatory medical device (wherein the information includes a plurality of cardiac depolarizations), generating an indication of correlation between each of at least a portion of the cardiac depolarizations and a stored template representative of a normal sinus rhythm, comparing the indications of correlation to a specified correlation threshold value, classifying, using the ambulatory medical device, the information representative of cardiac activity as a specific cardiac rhythm using the comparison of the indications of correlation to the specified correlation threshold value, identifying at least one indication of correlation that determines the classification, and providing the identified indication of correlation to a user or process.

In example 13, the subject matter of example 12 can optionally include detecting, with the ambulatory medical device, an arrhythmia using the information representative of cardiac activity. The classifying the rhythm can optionally include classifying the arrhythmia as supraventricular tachycardia (SVT) when a minimum of X of Y cardiac depolarizations satisfy the specified correlation threshold value, wherein X and Y are integers and X is less than Y, and the identifying an indication of correlation that determines the classification includes identifying the Xth highest indication of correlation to the user or process.

In example 14, the subject matter of one or any combination of claims 12 and 13 can optionally include detecting, with the ambulatory medical device, an arrhythmia using the information representative of cardiac activity. The classifying the arrhythmia optionally includes classifying the arrhythmia as ventricular tachycardia when a minimum of Z of Y cardiac depolarizations fail to satisfy the specified correlation threshold value, wherein Z and Y are integers and Z is less than Y, and the identifying an indication of correlation that determines the classification optionally includes identifying the Zth lowest indication of correlation to the user or process.

In example 15, the generating an indication of correlation of one or any combination of claims 12-14 can optionally include generating a first indication of correlation between a first cardiac depolarization and the stored template and generating a second indication of correlation between a second cardiac depolarization and the stored template. The subject matter can optionally include providing to a user or process, the first and second indications of correlation, the information representative of the first and second cardiac depolarizations, and information relating the first indication and the second indication to the first cardiac depolarization and the second cardiac depolarization respectively.

In example 16, the providing to a user or process of one or any combination of examples 12-15 can optionally include storing, for subsequent display, the first and second indications of correlation, the information representative of the first and second cardiac depolarizations, and the information relating the first indication and the second indication to the first cardiac depolarization and the second cardiac depolarization.

In example 17, the providing to a user or process of one or any combination of examples 12-16 can optionally include communicating, to a second device for display, the first and second indications of correlation, the information representative of the first and second cardiac depolarizations, and the information relating the first indication and the second indication to the first cardiac depolarization and the second cardiac depolarization.

In example 18, the subject matter of one or any combination of examples 12-17 can optionally include displaying the first and second indications using a second device, the displaying including substantially aligning the first indication with the representation of the first cardiac depolarization on the display and substantially aligning the second indication with the representation of the second cardiac depolarization on the display.

In example 19, the generating the first and second indications of correlation of one or any combination of examples 12-18 can optionally include calculating a first measure of correlation of the first cardiac depolarization to the stored template and calculating a second measure of correlation of the second depolarization to the stored template, and wherein the first and second indications of correlation include the first measured correlation and the second measured correlation respectively.

In example 20, the generating the first and second indications of correlation of one or any combination of examples 12-19 optionally includes calculating a first measure of correlation of the first cardiac depolarization to the stored template, and calculating a second measure of correlation of the second cardiac depolarization to the stored template. The comparing optionally includes comparing the first and second measures of correlation to the specified correlation threshold value. The first and second indications optionally include a result of the comparison of the first measured correlation and a result of the comparison of the second measured correlation respectively.

### Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, the code can be tangibly stored on one or more volatile or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A system, comprising:
an ambulatory medical device including:
a cardiac activity sensing circuit configured to obtain information representative of electrical cardiac activity of a subject, wherein the information includes a plurality of cardiac depolarizations;
a processing circuit communicatively coupled to the cardiac activity sensing circuit, wherein the processing circuit includes:
a correlation circuit configured to generate an indication of correlation between each of at least a portion of the cardiac depolarizations and a stored template representative of a normal sinus rhythm; and
a rhythm discrimination circuit configured to:
compare the indications of correlation to a specified correlation threshold value;
classify the information representative of cardiac activity as a specific cardiac rhythm using the comparison; and
identify at least one indication of correlation that determines the classification, and
wherein the processing circuit is configured to provide the identified indication of correlation to a user or process.

2. The system of claim 1, wherein the rhythm discrimination circuit is configured to:
detect an arrhythmia using the information representative of cardiac activity;
classify the arrhythmia as supraventricular tachycardia (SVT) when a minimum of X of Y cardiac depolarizations satisfy the specified correlation threshold value, wherein X and Y are integers and X is less than Y; and
identify the Xth highest indication of correlation as the indication that determines the classification.

3. The system of claim 1, wherein the rhythm discrimination circuit is configured to:
detect an arrhythmia using the information representative of cardiac activity;
classify the arrhythmia as ventricular tachycardia when a minimum of Z of Y cardiac depolarizations fail to satisfy the specified correlation threshold value, wherein Z and Y are integers and Z is less than Y; and
identify the Zth lowest indication of correlation as the indication that determines the classification.

4. The system of claim 1, wherein the correlation circuit is configured to:
generate a first indication of correlation between a first cardiac depolarization and the stored template;
generate a second indication of correlation between a second cardiac depolarization and the stored template, and
wherein the processing circuit is configured to provide, to a user or process: the first and second indications of correlation, the information representative of the first and second cardiac depolarizations, and information relating the first indication and the second indication to the first cardiac depolarization and the second cardiac depolarization respectively.

5. The system of claim 4, wherein the ambulatory medical device includes a memory circuit integral to, or communicatively coupled to, the processing circuit, wherein the memory circuit is configured to store, for subsequent display, the first and second indications of correlation, the information representative of the first and second cardiac depolarizations, and the information relating the first indication and the second indication to the first cardiac depolarization and the second cardiac depolarization.

6. The system of claim 4, wherein the ambulatory medical device includes:
a communication circuit communicatively coupled to the processing circuit, wherein the communication circuit is configured to communicate information with a second device, and
wherein the processing circuit is configured to communicate, to the second device for display, the first and second indications of correlation, the information representative of the first and second cardiac depolarizations, and the information relating the first indication and the second indication to the first cardiac depolarization and the second cardiac depolarization.

7. The system of claim 4, including a second device configured for communicating with the ambulatory medical device and including a display, wherein the second device is configured to display the first and second indications, including substantially aligning the first indication with the representation of the first cardiac depolarization on the display and substantially aligning the second indication with the representation of the second cardiac depolarization on the display.

8. The system of claim 4, wherein the correlation circuit is configured to calculate a first measure of correlation of the first cardiac depolarization to the stored template and calculate a second measure of correlation of the second depolarization to the stored template, and wherein the first and second indications of correlation include the first measured correlation and the second measured correlation respectively.

9. The system of claim 4, wherein the correlation circuit is configured to:
calculate a first measure of correlation of the first cardiac depolarization to the stored template;
calculate a second measure of correlation of the second cardiac depolarization to the stored template;
compare the first and second measures of correlation to a specified correlation threshold value, and
wherein the first and second indications include a result of the comparison of the first measured correlation and a result of the comparison of the second measured correlation respectively.

10. The system of claim 1, including a second device configured for communicating with the ambulatory medical device and including a display, wherein the rhythm discrimination circuit is configured to apply at least one rhythm detection enhancement method to classify the rhythm, and wherein the second device is configured to display the identified indication of correlation with one or more of the rhythm classification and a result of the rhythm detection enhancement method.

11. The system of claim 1, wherein the ambulatory medical device includes a communication circuit communicatively coupled to the processing circuit, wherein the communication circuit is configured to receive an updated correlation threshold from a second device, and
wherein the correlation circuit is configured to use the updated correlation threshold as the specified correlation threshold for subsequent correlation determination.

12. A method comprising:
obtaining information representative of electrical cardiac activity of a subject using an ambulatory medical device, wherein the information includes a plurality of cardiac depolarizations;
generating an indication of correlation between each of at least a portion of the cardiac depolarizations and a stored template representative of a normal sinus rhythm;
comparing the indications of correlation to a specified correlation threshold value;
classifying, using the ambulatory medical device, the information representative of cardiac activity as a specific cardiac rhythm using the comparison of the indications of correlation to the specified correlation threshold value;
identifying at least one indication of correlation that determines the classification; and
providing the identified indication of correlation to a user or process.

13. The method of claim 12, including:
detecting, with the ambulatory medical device, an arrhythmia using the information representative of cardiac activity;
wherein classifying the rhythm includes classifying the arrhythmia as supraventricular tachycardia (SVT) when a minimum of X of Y cardiac depolarizations satisfy the specified correlation threshold value, wherein X and Y are integers and X is less than Y; and
wherein identifying an indication of correlation that determines the classification includes identifying the Xth highest indication of correlation to the user or process.

14. The method of claim 13, including:
detecting, with the ambulatory medical device, an arrhythmia using the information representative of cardiac activity;
wherein classifying the arrhythmia includes classifying the arrhythmia as ventricular tachycardia when a minimum of Z of Y cardiac depolarizations fail to satisfy the specified correlation threshold value, wherein Z and Y are integers and Z is less than Y; and
wherein identifying an indication of correlation that determines the classification includes identifying the Zth lowest indication of correlation to the user or process.

15. The method of claim 12, wherein generating an indication of correlation includes:
generating a first indication of correlation between a first cardiac depolarization and the stored template;
generating a second indication of correlation between a second cardiac depolarization and the stored template, and
wherein the method includes providing to a user or process, the first and second indications of correlation, the information representative of the first and second cardiac depolarizations, and information relating the first indication and the second indication to the first cardiac depolarization and the second cardiac depolarization respectively.

16. The method of claim 15, wherein the providing to a user or process includes storing, for subsequent display, the first and second indications of correlation, the information representative of the first and second cardiac depolarizations, and the information relating the first indication and the second indication to the first cardiac depolarization and the second cardiac depolarization.

17. The method of claim 15, wherein the providing to a user or process includes communicating, to a second device for display, the first and second indications of correlation, the information representative of the first and second cardiac depolarizations, and the information relating the first indication and the second indication to the first cardiac depolarization and the second cardiac depolarization.

18. The method of claim 17, comprising:
displaying the first and second indications using a second device, the displaying including substantially aligning the first indication with the representation of the first cardiac depolarization on the display and substantially aligning the second indication with the representation of the second cardiac depolarization on the display.

19. The method of claim 15, wherein generating the first and second indications of correlation includes calculating a first measure of correlation of the first cardiac depolarization to the stored template and calculating a second measure of correlation of the second depolarization to the stored template, and wherein the first and second indications of correlation include the first measured correlation and the second measured correlation respectively.

20. The method of claim 15, wherein generating the first and second indications of correlation includes:
calculating a first measure of correlation of the first cardiac depolarization to the stored template; and
calculating a second measure of correlation of the second cardiac depolarization to the stored template,
wherein the comparing includes comparing the first and second measures of correlation to the specified correlation threshold value, and
wherein the first and second indications include a result of the comparison of the first measured correlation and a result of the comparison of the second measured correlation respectively.
